(19) United States
(12) Patent Application Publication
(10) Pub. No.: US 2011/0256209 A1
(43) Pub. Date: Oct. 20, 2011

(54) Skin patch for improving menstrual

(76) Inventor: Yon Doo Ough, Beloit, WI (US)

(21) Appl. No.: 13/134,919

(22) Filed: Jun. 20, 2011

Related U.S. Application Data
(63) Continuation-in-part of application No. 11/316,053, filed on Dec. 21, 2005.
Application No. 11/316,053 is a continuation-in-part of application No. 11/059,857, filed on Feb. 17, 2005, now abandoned.
(60) Provisional application No. 60/730,484, filed on Oct. 26, 2005.
(60) Provisional application No. 60/748,895, filed on Dec. 8, 2005.

(51) Int. Cl. A61K 9/70; A61P 15/00; A61K 36/61

(52) U.S. Cl. 424/447; 424/742

(57) ABSTRACT

A patch and topical application for improving discomfort from menstruation includes soap scented oil mixed with vegetable oil, such as castor oil.

### CROSS REFERENCE TO RELATED APPLICATIONS

The present application is a continuation-in-part of co-pending U.S. application Ser. No. 11/316,053 filed on Dec. 21, 2005, which claimed the benefits of U.S. Provisional Application No. 60/730,484 filed on Oct. 26, 2005 and U.S. Provisional Application No. 60/748,895 filed on Dec. 8, 2005; and which was a continuation-in-part of co-pending U.S. application Ser. No. 11/059,857 filed on Feb. 17, 2005.

### FIELD OF THE INVENTION

The present invention generally relates to the treatment of physical discomforts, and, more specifically, to devices applied and methods to improve discomfort experienced during menstruation.

### BACKGROUND OF THE INVENTION

Improving discomfort of various types can improve the quality of life for individuals who experience the discomfort. Some individuals experience event-specific discomfort. Others experience recurring discomfort, which may be somewhat continuous, or may involve repetitious intervals of discomfort following periods that are more or less discomfort-free. Menstruation can cause days of significant discomfort on a monthly basis for many years. Chronic conditions, such as irritable bowel syndrome, can be intermittent, but significant when the symptoms are manifested. Surgeries and injuries can result in long periods of discomfort for some individuals. Various back conditions can cause almost constant discomfort, as can arthritis and other joint conditions common in older people, making daily life difficult. Intense discomfort can be debilitating. Less intense discomfort, if frequent, can alter an individual's life-style.

Treatment protocols have changed and evolved. Cultural differences have begun to breakdown as medical professionals look for effective treatments for various patients experiencing different types of conditions. Drug therapies are used widely and are effective for many conditions. However, some patients and some conditions do not respond well to drug treatments. Complementary and alternative treatments, acupuncture, folk medicine remedies, mind-control and other treatments all have been used effectively by some individuals. Often, the effectiveness can not be explained medically or scientifically, even though the effectiveness is clearly evident and documented by the response of patients. One such preventative treatment has suggested that a bar of soap be placed near the legs while sleeping to prevent the onset of leg cramps while sleeping.

Castor oil has been reported as a successful alternative medicine when applied directly to and rubbed into an affected area; however, successful use of castor oil has been inconsistent and not well-understood. Some have suggested that success is psychological more than therapeutic. At best, its effect has not been well-understood.

One of the difficulties in treating discomfort is that the sources vary and may not be well understood. Athletes often experience recurring discomfort from intense use or over-use of muscles. For example, distance runners often experience chronic calf and thigh muscle discomfort. Many females regularly experience various degrees of discomfort for several days in their menstrual cycles. Conditions such as irritable bowel syndrome can be manifested by prolonged discomfort. The causes are not well understood and effective treatments are not readily available for all who suffer the symptoms. Conditions such as fibromyalgia and multiple sclerosis can cause general body discomfort that is more severe in some areas than in others. Widely effective treatments are not known. Many different medications are used with inconsistent results.

Analgesic treatments can be used to mask pain and discomfort, but are effective for only a limited duration of time and therefore must be repeated to provide continuous relief. Muscle relaxants provide some relief for muscle spasms, but also must be repeated. Continuous, prolonged use of drugs can result in undesirable side effects, including a build up of tolerance to the drug, requiring increased dosages for long term treatment. Addiction can occur rapidly and even predictably with some drugs, requiring very careful drug use management and withdrawal treatments when the drug use is ended. Other side effects can include possible organ functional changes and/or damage, mental and personality changes, changes to sensory perceptions and the like.

What is needed is a safe and effective treatment for a variety of discomfort that is easy to use, suitable for self-administration.

### SUMMARY OF THE INVENTION

The present invention provides a treatment for human discomfort which includes the application of a scented skin patch on the afflicted area. A diluted soap scented oil is used. The soap scent oil may be diluted with vegetable or other oils. Scent oil dilutions with castor oil provide advantages for some treatments.

Other features and advantages of the invention will become apparent to those skilled in the art upon review of the following detailed description, claims and drawings in which like numerals are used to designate like features.

### BRIEF DESCRIPTION OF THE DRAWINGS

FIG. 1 is a plan view of a patch suitable for treatments in accordance with the present invention;

FIG. 2 is a cross-sectional view of a patch similar to that shown in FIG. 1, the cross-section having been taken along line 2-2 as shown on the patch of FIG. 1;

FIG. 3 is a cross-sectional view similar to FIG. 2, but illustrating a further embodiment of the present invention;

FIG. 4 is a cross-sectional view similar to FIGS. 2 and 3, but illustrating a further embodiment of the present invention;

FIG. 5 is a perspective view of another patch; and

FIG. 6 is a cross-sectional view of the patch shown in FIG. 5.

Before the embodiments of the invention are explained in detail, it is to be understood that the invention is not limited in its application to the details of construction and the arrangements of the components set forth in the following description or illustrated in the drawings. The invention is capable of other embodiments and of being practiced or being carried out in various ways. Also, it is understood that the phraseology and terminology used herein are for the purpose of description and should not be regarded as limiting. The use herein of “including”, “comprising” and variations thereof is meant to encompass the items listed thereafter and equivalents thereof, as well as additional items and equivalents thereof.

### DETAILED DESCRIPTION OF THE PREFERRED EMBODIMENT

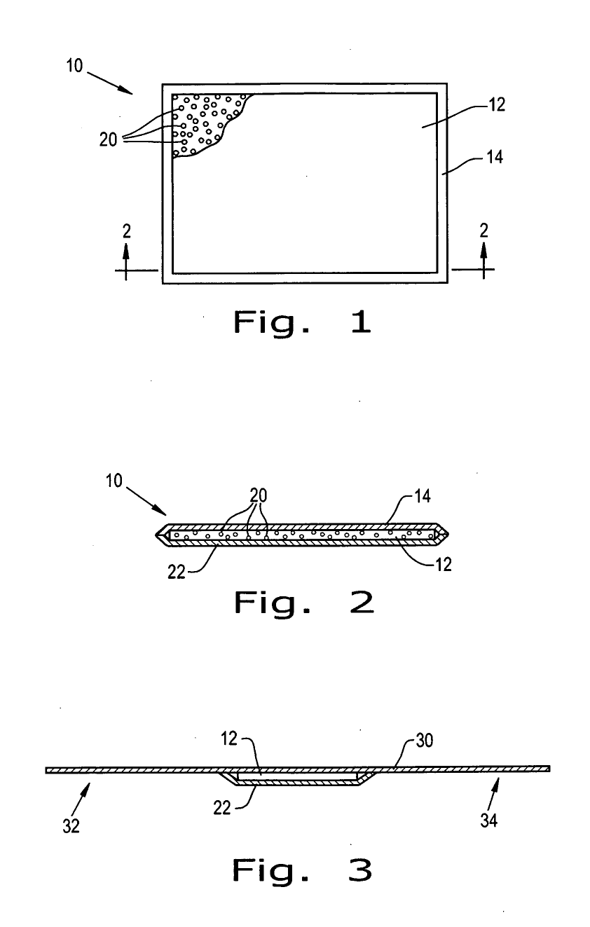

Referring now more specifically to the drawings and to FIG. 1 in particular, numeral 10 designates a patch useful to improve discomfort during menstruation and other conditions. Patch 10 includes an application layer 12 and a cover 14. Patch 10 can be provided in a variety of shapes and sizes for use on different parts of the body for different purposes.

Application layer 12 is a pad of cotton gauze or other material suitable for retaining scent oil and a dilution oil. Application layer 12 is provided with a product having the scent of soap, which may be soap scented oil, or fragments, chips, shavings or other small pieces of soap dispersed through, along and against application layer 12. For simplicity and clarity in the drawings, substance 20 is illustrated only in a corner region of application layer 12; however, it should be understood that substance 20 is relatively evenly distributed throughout application layer 12, in the preferred arrangement. Further, for clarity, only some, and not all of the illustrated substance 20 elements are designated with a reference numeral in FIG. 1

As used herein, including in the claims, the term “soap” is meant to include simple or “pure” soaps made from fat and an alkali in a standard saponification or hydrolysis reaction. Scents of soaps made from sodium hydroxide and fat are preferred. While soaps including other additives can be used, a simple or substantially “pure” soap consisting primarily of the product resulting from the saponification of a fat and alkali is preferred, without excessive amounts of perfumes, colorants, germicides, lotions or other conditioners having been added. Hand soap of a bar type manufactured by Procter & Gamble marketed under the name IVORY® (Original Blend) has been used effectively in the present invention. Soaps made from fat and other alkalis, such as potassium hydroxide may also be suitable; however, the aforementioned IVORY® soap has been found particularly effective.

Scent of soaps of the type used for hand and human body washing are preferred. Such soaps have been found to work effectively in relieving discomfort, cramps, spasms and a variety of other ailments and appear to be safe even when placed in close proximity to a treated area for a prolonged period of time.

While actual soap fragments or pieces can be used as substance 20, an increased effect has been observed when soap scented oil is used. A scent oil of the aforementioned IVORY® soap was obtained and diluted. Application layer 12 was substantially saturated with the scent oil dilution and used effectively. Thus, it is intended that the elements 20 shown in FIG. 1 can be fragments of soap within or on application layer 12, as well as droplets of soap scent oil or a scent oil dilution held or absorbed by application layer 12. It is believed that other constructions can be used including impregnation of a carrier layer with a suitable substance 20 such as gel or other liquid, solid or semi-solid having the scent of soap.

Cover layer 14 is a substantially impermeable fabric or other material covering one side of application layer 12 and substance 20 contained therein. Cover 14 is slightly larger than application layer 12, and helps confine the scent, sublimates or vaporization products from substance 20 to the area upon which patch 10 is applied. Cover 14 can be provided with an adhesive coating for securing cover layer 14 to application layer 12.

Patch 10 can be used as thus far described, with the exposed side of application layer 12 placed against the skin, and cover layer 14 exposed on the outer side of patch 10. Patch 10 can be secured in the desired position on the skin using adhesive tape or other binding materials in a manner similar to the application and fixation of wound dressings.

For further integrity of patch 10, a transmission layer 22 can be used on the inwardly facing surface of application layer 12. In the exemplary embodiment, transmission layer 22 is a layer of breathable adhesive tape extending beyond application layer 12 to adhere to cover layer 14 about the periphery of application layer 12. Transmission layer 22 is a material through which the scent, sublimates or vaporization products of substance 20 pass readily. Thus, as illustrated in FIG. 2, application layer 12 containing substance 20 is confined within a pocket formed by substantially impermeable cover layer 14 on one face thereof and by permeable transmission layer 22 on the other side thereof.

In a further embodiment shown in FIG. 3 application layer 12, containing substance 20, is attached to an elongated web 30, which has tail end portions 32, 34 extending beyond opposite sides of application layer 12. The aforementioned transmission layer 22 of breathable tape can be used for attaching application layer 12 to web 30. Web 30 can be an adhesive tape or other substantially impermeable material functioning similarly to cover layer 14 for confining the scent from substance 20. Alternatively, a cover layer 14 can be interposed between application layer 12 and web 30.

Tails 32, 34 are used to facilitate securing application layer 12 in the desired position. Thus, tails 32, 34 can be provided with a coating of suitable adhesive on the surface thereof with application layer 12, so that tails 32, 34 can be adhered to the skin of a person using the present invention. The length, shape and other size of tails 32, 34 can be varied to provide patches 10 of different configurations for use on various parts of the human body. Further, elongated tails 32, 34 without adhesive also can be used. Web 30 can be in the nature of an elastic bandage or other binder material to wrap the leg, arm or torso of the person using it. Non-adhesive tails 32, 34 can be secured to each other or to the person by commonly used adhesive tape, mechanical fastener or other securing devices. Instead of tails, a complete border surrounding application layer 12 can be used, with or without adhesive.

In using patch 10, the area of discomfort is identified, and patch 10 is placed substantially centrally thereon to bring substance 20 in close proximity to the area. Patch 10 is secured in place via tape, tails 32, 34 or suitable means. Patch 10 should be kept on the area being treated for at least six to eight hours, or until such time as the discomfort improves. While patch 10 can be worn substantially continuously, effective treatment of many discomforts has been observed when patch 10 is used for approximately six to eight hours during successive twenty-four hour periods. Thus, for many discomforts, effective treatments can be performed with nighttime applications of patch 10, while the user is resting or asleep and not overly active.

A scent oil including at least about 10% citronella oil; about 5-10% each of benzyl benzoate, citronellal, and geraniol; and about 1-5% each of benzophenone, camphor, citronellol, phenoxybenzene, eucalyptus globulus leaf oil, thymol and dodecanal have been used diluted with vegetable oil or castor oil. These have been further mixed with 0.8% menthol. Patches as illustrated by patch 100 were made with so-called standard IVORY® fragrances from Belle Aire Fragrances, Inc. of Mundelein, Ill. and from Ciaroma supplied through Scentoils.com. Scent oils are mixtures of natural oil scents, essential oils and proprietary components of each supplier. The scent oils were diluted with a high-grade vegetable oil recommended and supplied by Belle Aire. Solutions of 3%, 5%, 10% and 20% scent oil, with the remainder being vegetable oil were used. Still other types of oil also can be used, such as coconut oil and other oils used for personal hygienic products and cosmetics. For treating some conditions such as arthritis and other joint discomforts, pure castor oil from Now Foods, Bloomingdale, Ill. 60108, was used to dilute the scent oil. FIG. 4 illustrates a patch 100 used for the aforedescribed dilutions. The selected diluted solution, using vegetable oil or castor oil was sprayed on a Kendall brand (CURITY®) sponge pad 102. Pad 102 was placed on an impervious backing 104 cut from a thick, high quality surgical drape. Three types of 3M brand medical tapes of different porosity (marketed as MEDIPORE®, TRANSPORE® and DURAPORE®) were used as a cover 106, an edge seal 108 and edge reinforcement 110, respectively. It should be understood that diluents other than vegetable oils and castor oil may also be useful.

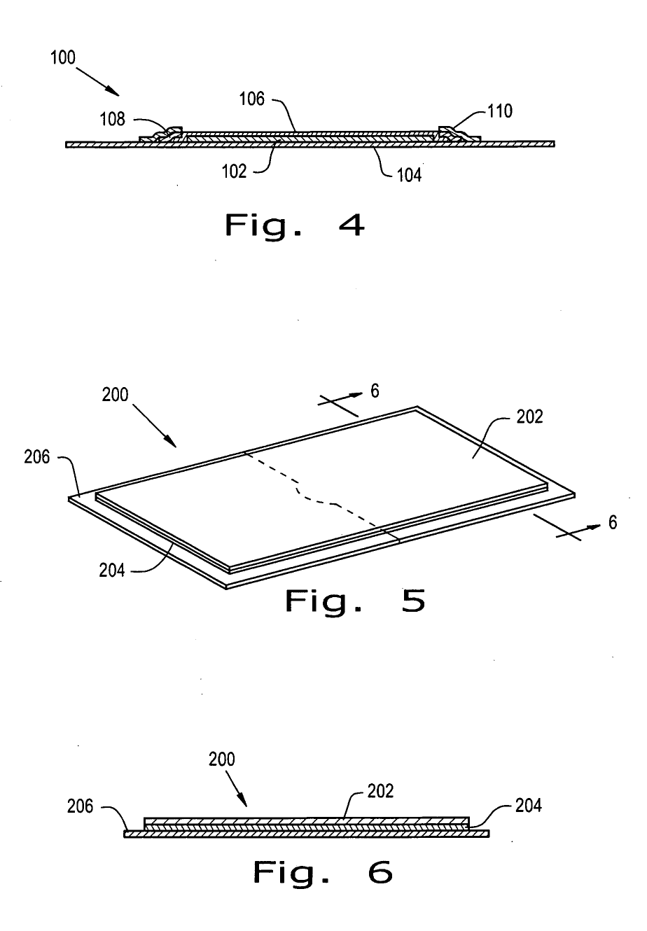

FIGS. 5 and 6 illustrate yet another embodiment of a skin patch 200 manufactured by a so-called dry technique. As provided for use, skin patch 200 includes a removable protective layer 202 over a treatment layer 204 provided on a backing layer 206. Treatment layer 204 includes the aforedescribed scent oil mixed with an acrylic adhesive solution that is applied to backing layer 206 and thereafter dried. After drying, the acrylic adhesive remains sticky for direct adhesion to skin. Removable protective layer 202 covers and protects the exposed surface of treatment layer 204 prior to use. When patch 200 is to be used, protective layer 202 is removed and treatment layer 204 is placed against the skin. To facilitate removal of protective layer 202, a divide or cut 208 can be provided in the protective layer so that it can be removed in separate pieces. The adhesive nature of treatment layer 204 causes the patch to self adhere to the skin without the need for additional tape or other apparatus for holding patch 200 in place. In this construction, patch 200 can be provided as a very thin device having only a relatively thin backing layer 206 of cloth with a thin coating comprising treatment layer 204. Accordingly, patch 200 can be worn inconspicuously underclothing and the like as a person goes about routine daily activities, and if backing layer 206 is provided in natural skin colors any exposed portions of patch 200 are relatively inconspicuous. Further, the patch is convenient to carry in a sealed envelope or sleeve, for application and use whenever needed.

The aforedescribed scent oil also can be mixed with a standard cream base or balm to make a topical application that can be applied by hand and rubbed into the skin. Applied in this manner, a patch or other covering is not required. The cream can be applied whenever necessary, and can be carried conveniently in a tube or jar.

The present invention has been tried on a variety of individuals experiencing different types of general discomforts. The observed effectiveness is reported below.

Multiple individuals who experienced severe menstrual discomfort every month for many years used patches of the present invention made with scent oil diluted with vegetable oil. With consistent use, improvement has been reported each month. Applying a patch at the onset of discomfort has provided very rapid improvement for all individuals, and further discomfort is avoided with continued use during the approximately two days of the menstrual cycle during which discomfort had been experienced in the past. Generally, improvement of menstrual discomfort is experienced within about thirty minutes after applying a patch.

One individual reported experiencing severe menstrual discomfort for many years. On a first day of severe, almost intolerable discomfort, a patch was applied with initial significant improvement. A patch was worn continuously for two days, with significant to complete improvement reported.

A marathon runner experienced frequent cramps, tenderness and generally achy sensations in calf muscles for more than ten months. Palpable knots in the calf muscle were present. Physical therapy treatments, including ultra-sound treatments, acupuncture and massage provided little relief. A patch as described herein was placed on the calf muscle. After two days, all symptoms were reported to be gone. During running a marathon, a patch was placed on the calf muscle of one leg, but not the other. The leg without a patch experienced discomfort. The leg with a patch of the present invention thereon experienced no similar discomfort.

An individual experienced severe myofascial discomfort (so called “trigger point pain”) and tightening in a shoulder for more than two years. Trigger point injections of local anesthetic, steroid treatment and botox injections provided only short-term relief. A patch was applied to the area. The person reported fifty-percent improvement after one day and eighty-percent improvement after two days, with all discomfort relieved after five days.

A second individual experienced myofascial discomfort in a shoulder for over one year following a sports-related injury. Standard treatments were not effective. With consistent use of a patch as described herein, improvement was reported.

A third individual experienced myofascial discomfort between the shoulder blades for five days. A patch was applied on the tender area for two successive evenings and resulted in improvement of the discomfort.

An individual experienced severe neck and shoulder discomfort and tightness following radical neck surgery and radiation treatments for cancer. The discomfort was unresponsive to all treatments tried, including physical therapy, drugs, yoga, massage, heating pads, hot soaks, weight training and the use of special apparatus. A patch as described herein was applied to the area overnight. After five successive treatments, eighty-percent improvement in the discomfort was reported and was maintained with continued applications of a patch overnight.

An individual suffering discomfort from a laparoscopic tubal ligation applied a patch over the umbilicus and reported generally feeling better, with no discomfort.

An individual with a bulging disc for two months received acupuncture and chiropractic treatments with only temporary relief An epidural steroid injection provided no relief after three days. A patch as described herein was applied with a heat pack for 30 minutes, and thereafter without heat. After three days, fifty percent improvement was reported.

An individual reported irritable bowel syndrome for over fifty years, with worsening conditions. Symptoms included excessive gas, explosive bowel movements, frequent urge and bowel movements (seven or eight times daily), and very loose stools with frequent diarrhea. Daily treatments of prescribed and over-the-counter medicines, and dietary changes provided little relief A patch first with fragments 20 and later with diluted scent oil 20 was applied over the lower abdomen while sleeping. Gradual improvement of symptoms was reported over the first week. All other medications were stopped, and eighty-percent improvement in symptoms was reported. Gas, looseness of stools and diarrhea were gone, and bowel movement urges were reduced to three or four daily.

A forty-four-year-old female with a long history of irritable bowel syndrome had frequent abdominal spasmodic and cramping discomfort. A patch on a binder was applied to the lower abdomen and relieved cramping discomfort completely. After a period of using a patch, all other medications were discontinued

A fifty year old male reported stiffness in the neck upon awakening. A patch was applied over the affected area. Within one hour the condition lessened and was completely relieved within one day. The improvement was only temporary, and stiffness returned on the second day. The patch was repositioned to an area seemingly more affected, and further improvement was experienced.

Individual 28 and 55 years old suffering achy discomfort from fibromyalgia have used the scent oil patches daily for constant improvement.

Patch 10 has manifested effectiveness in improving occurrences of muscle cramping, muscle spasms and discomfort from a number of sources and conditions. It is believed that other, discomforts, spasms and cramps might be equally responsive to treatment by patch 10. Conditions that respond to the present invention appear to be those for which improvement can be obtained by treating the symptom without treatment of the underlying condition causing the symptom, with the condition being treated otherwise, or simply healing or entering states of remission.

The mechanism of improvement is not completely understood; however, there appears to be a relationship to the scent or components thereof and an apparent transdermal transmission thereof to the area being treated. Several users have reported an improved therapeutic result when using a patch of the present invention made with scent oil, when compared to using fragments of soap, leading to a belief that components of scent are involved. Whatever the mechanism, a significant improvement has been achieved by placing the scent of soap in close proximity to the area of discomfort and maintaining the close proximity position for an extended time, such as about at least six to eight hours during a twenty-four hour time period.

A summary of the observed and anticipated conditions that can be improved the use of the patches described herein includes very effective results observed in improving discomforts associated with menstruation, with no failures in obtaining improvement. Results have been obtained quickly, often in a matter of a few minutes. It is believed from the observed results in treating menstrual discomforts that the patches could be used to treat other discomforts associated with uterine contractions, such as the onset of premature labor. Advantages would be obtained over the use of medicines currently used that have systemic effects.

Treatment of skeletal muscle discomfort associated with spasms, cramps, tightening, knots and stiffening, including discomfort from multiple sclerosis. Successfully improved conditions include discomforts associated with strenuous exercise, hard and/or prolonged labor, trauma, poor positioning, posture or work conditions, seemingly spontaneously occurring neck stiffness, fibromyalgia, surgery, and herniated discs.

Based on observations, smooth muscle discomforts are consistently improved more effectively and more easily than skeletal muscle discomforts. More effective treatment is realized with higher concentrations of scent oil.

Treatment of discomforts of the digestive system, and particularly those resulting from abdominal cramps with intestinal spasms. Successfully treated discomforts have resulted from spasmodic bowel disease, chronic indigestion, excessive gas, and irritable bowel syndrome.

Since the patches have exhibited effectiveness in treating smooth muscle discomforts, it is believed effectiveness will occur in the treatment of other types of smooth muscle discomforts, such as those resulting from arterial spasm, which may be brought on by vascular surgery, or arterial spasmodic conditions. This may include spasmodic angina.

A variety of smooth muscle discomforts resulting from spasmodic conditions of the abdomen should also respond to the present treatment, including those resulting from gall bladder disease, ureteral stones, and urinary bladder spasms.

The aforedescribed uses by individuals supports at least several conclusions. Scent of soap has an improvement effect for several conditions when the soap is placed in close proximity to the affected area and retained there for an extended time period. Components of soap scent have an improvement effect when applied to the affected area. Scent of soap can be administered to treat smooth muscles discomfort of internal organs. These conclusions, and particularly the indications that smooth muscles discomforts of internal organs can be improved by the application of a patch on the skin near the organ, has implications for application with other drugs and medications beyond the use of soap and soap scent products as described herein.

As mentioned previously herein, the aforementioned scent oils have been diluted with castor oil and used with constructions in the manner of patch 100. Patches made with castor oil have been used to improve discomfort from degenerative arthritis and other joint conditions with great success. Conditions treated include arthritic patients, including an individual with end-stage osteoarthritis in both ankles that had been treated with fusion surgery. Using patches made with scent oil and castor oil, discomfort was improved in both ankles, with greater improvement more quickly in one ankle than the other. An individual with end-stage osteoarthritis in a foot used significant daily dosages of narcotics for pain relief. After one week using a patch made from scent oil and castor oil the discomfort was relieved. An individual with arthritis in both thumbs was treated with a small patch and experienced improvement. Undiagnosed knee discomfort believed to be arthritic in nature was improved.

A young female with annoying knee discomfort that interrupted nighttime sleep was relieved of all discomfort following one weekend treatment with a patch of the present invention.

An individual with spinal stenosis and symptoms of sciatica experienced improvement following treatment with a patch made with scent oil and castor oil.

A runner with foot discomfort diagnosed as a bruised tendon reported great improvement from use of a patch made with scent oil and castor oil.

Patients having chronic knee joint discomfort from degenerative arthritis experienced improvement using a patch made with castor oil and scent oil.

Three patients age 50, 56 and 62 diagnosed with tennis elbow (tendinitis) applied scent oil patches diluted with castor oil and experienced improvement.

Variations and modifications of the foregoing are within the scope of the present invention. It is understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention. The claims are to be construed to include alternative embodiments to the extent permitted by the prior art.

Various features of the invention are set forth in the following claims.

What is claimed is:

1. A topical application for improving discomfort from menstruation, comprising:
a scent oil including at least camphor and eucalyptus globulus leaf oil and at least one of citronella oil, citronellal and citronellol; and
a vegetable oil mixed with said scent oil.

2. The topical application of claim 1, including less than 1% concentrations of said camphor and said eucalyptus globulus leaf oil.

3. The topical application of claim 2, said vegetable oil being castor oil.

4. The topical application of claim 2, further including menthol in a concentration of less than 1%.

5. The topical application of claim 4, said vegetable oil being castor oil.

6. The topical application of claim 1, said scent oil including at least about 10% citronella oil; between about 5% and 10% each of benzyl benzoate, citronellal, and geraniol; and between about 1% and 5% each of benzophenone, camphor, citronellol, phenoxybenzene, eucalyptus globulus leaf oil, thymol and dodecanal.

7. The topical application of claim 6, including between about 3% and 20% of said scent oil by volume.

8. The topical application of claim 7, further including menthol in a concentration of less than 1%.

9. The topical application of claim 7, said vegetable oil being castor oil.

10. The topical application of claim 9, further including menthol in a concentration of less than 1%.

11. The topical application of claim 10, further comprising a patch transporting said sent oil and vegetable oil and adhesive for holding the patch on human skin.

12. The topical application of claim 1, further comprising a patch transporting said sent oil and vegetable oil and adhesive for holding the patch on human skin.

13. An application to improve discomfort associated with menstruation, comprising:
a mixture including vegetable oil and between about 3% and about 20% scent oil by volume; and
said scent oil including at least about 10% citronella oil; between about 5% and 10% each of benzyl benzoate, citronellal, and geraniol; and between about 1% and 5% each of benzophenone, camphor, citronellol, phenoxybenzene, eucalyptus globulus leaf oil, thymol and dodecanal.

14. The application of claim 13, said mixture including menthol.

15. The application of claim 13, said vegetable oil being castor oil.

16. The application of claim 15, said mixture including menthol.

17. The application of claim 15, said mixture further combined with an adhesive and applied to a fabric backing.

18. The application of claim 13, said mixture further combined with an adhesive and applied to a fabric backing.
